(19) United States
(12) Patent Application Publication
(10) Pub. No.: US 2008/0195195 A1
(43) Pub. Date: Aug. 14, 2008

(54) Detectable Stent and Method of Making Same

(76) Inventor: Henry W. Lupton, Galway (IE)

Correspondence Address: Allen W. Wark;Abbott Laboratories, D377/AP6A-1, 100 Abbott Park Road, Abbott Park, IL, 60064-6008, US

(21) Appl. No.: 10/490,680

(22) PCT Filed: Sep. 23, 2002
(86) PCT No.: PCT/GB02/04318

§ 371 Date: Aug. 7, 2006

(30) Foreign Application Priority Data
Sep. 24, 2001 (GB) 0122965.7

(51) Int. Cl. A61F 2/82; A61F 2/06

(52) U.S. Cl. 623/1.34; 29/428

(57) ABSTRACT

A stent includes marker sections (5) having thicknesses greater than that of the remainder of the stent so that the marker sections (5) become radiopaque and visible to a surgeon deploying the stent in use. The stent is formed by producing apertures in a hollow tube such that the tube becomes a series of interconnecting struts defining the apertures (2), with the marker sections (5) formed by folding at least one portion of the wall to provide a region of increased thickness. The marker sections (5) may be formed at the ends of the stent and/or at an intermediate area of the stent away from the ends. The marker sections may be formed by folding the at least one portion of the wall to the extent that the original outer surface of the portion is adjacent to the outer surface of the wall such that a two laywer region of wall is provided as a marker section (5). The marker sections (5) may be formed by folding the at least one portion of the wall into three layers. The folded portions of the marker sections may also be secured by welds.

### FIELD OF THE INVENTION

The present invention generally relates to stents. Particularly, it relates to expandable stents, such as balloon expandable stents. Such expandable stents are formed by producing apertures in a hollow tube such that the tube becomes a series of interconnecting struts defining the apertures. The tube can then be inserted, in use, into a body lumen, such as an artery, and expanded when in use to hold open that lumen. The expansion is usually performed by application of expansive pressure to the tube by inflation of a balloon or similar device.

### BACKGROUND FO THE INVENTION

In recent years there have been many developments in the technology associated with producing such stents such that it is now possible to produce stents of extremely small diameter for use in small lumens and such that it is possible to produce stents with extremely narrow struts between apertures to increase the flexibility of the stent when in its unexpanded state in order to ease delivery to a desired site. A significant disadvantage of these developments is, however, that the stents have become less visible to a surgeon. Usually, in order to determine the location of the stent during delivery, the body lumens being treated are viewed by a surgeon under x-ray or similar technique. For stents of reduced diameter and reduced strut material it can become difficult for the stent itself to be viewed as it becomes less and less radiopaque the less material it contains. This can make delivery of such stents an extremely complex task.

### SUMMARY OF THE INVENTION

The present invention seeks to provide a stent which has increased radiopacity, yet which can still be produced to extremely small diameters and have support struts which are small enough to allow a high degree of flexibility in the stent. Furthermore, it seeks to provide a stent which will not break up during expansion and which will not have components which break off readily causing damage to the recipient.

According to the present invention there is provided a stent comprising a tubular wall with apertures formed therein to allow for expansion of the stent, in use, by outward pressure applied to the stent, the stent further comprising marker sections, the marker sections being formed by folding at least one portion of the wall to provide a region of increased thickness.

The marker sections may be formed only at the ends of the stent. The marker sections may be formed by folding the at least one portion of the wall to the extent that the original outer surface of the portion is adjacent to the outer surface of the wall such that a two layer region of wall is provided as a marker section. The marker sections may be formed by folding the at least one portion of the wall into three layers. The folded portions of the marker sections may be secured by welds.

The present invention also provides a method of manufacturing a stent comprising a tubular wall with apertures formed therein to allow for expansion of the stent, in use, by a balloon, the method including the steps of providing the stent, providing a die which is capable of presenting a series of inclined surfaces to at least one portion of the stent wall, forming a contact between the at least one portion of the stent wall and the die, and progressively folding the portion of the stent wall to provide a marker section of increased wall thickness by generating pressure on the contact.

The die may comprise multiple components with a different inclined surface being provided by each component, such that the contact and folding steps further includes the steps of presenting the stent to a first component of the die, generating pressure at the stent contact to force the stent to take the shape of the component, and repeating these steps at each component, in turn, to progressively form the folds of the marker sections.

In the above case, the components of the die may be a series of protrusions, the base of each protrusion comprising a molded region, wherein the shapes of these molded regions are sequentially shaped to progressively fold the wall region when the stent is pressed onto them in turn, with the method further including the steps of providing a housing to support the stent, and wherein the step of presenting the stent to the die further includes the steps of placing the stent, surrounded by its housing, over one of the protrusions of the die, introducing one end of the stent to the base of the protrusion, generating pressure at the contact view to the housing such that the end of the stent is forced to take the shape of the base of the protrusion, and removing the stent and housing from the protrusion and placing it over the next one. Further, the method may include the additional step of welding the folded portions of the marker sections in place.

The present invention provides a stent which has markers which cover thickness which is greater than that of the remainder of the stent so that the markers become radiopaque and visible to a surgeon deploying the stent in use. Because the markers are formed from the wall of the stent there is a reduced likelihood of the markers breaking free and falling into the lumen of a recipient when installed. Furthermore, because the markers do not affect the structural integrity of the stent they can be employed with just about any expandable stent design that is currently in use so that stents of reduced diameter and high flexibility can have increased radiopacity by employing the concepts of the present invention.

### BRIEF DESCRIPTION OF THE DRAWINGS

Other advantages of the present invention will be readily appreciated and apparent to those skilled in the art as the same becomes better understood by reference to the following detailed description when considered in connection with the accompanying drawings, in which:

FIG. 1 is a perspective view of a stent which may employ the concepts of the present invention;

FIGS. 2 to 6 are schematic views of markers that may be produced in accordance with the present invention; and

FIGS. 7A to 7C are schematic diagrams showing an example method for producing the stent of FIGS. 1 to 6.

### DETAILED DESCRIPTION OF THE PREFERRED EMBODIMENTS

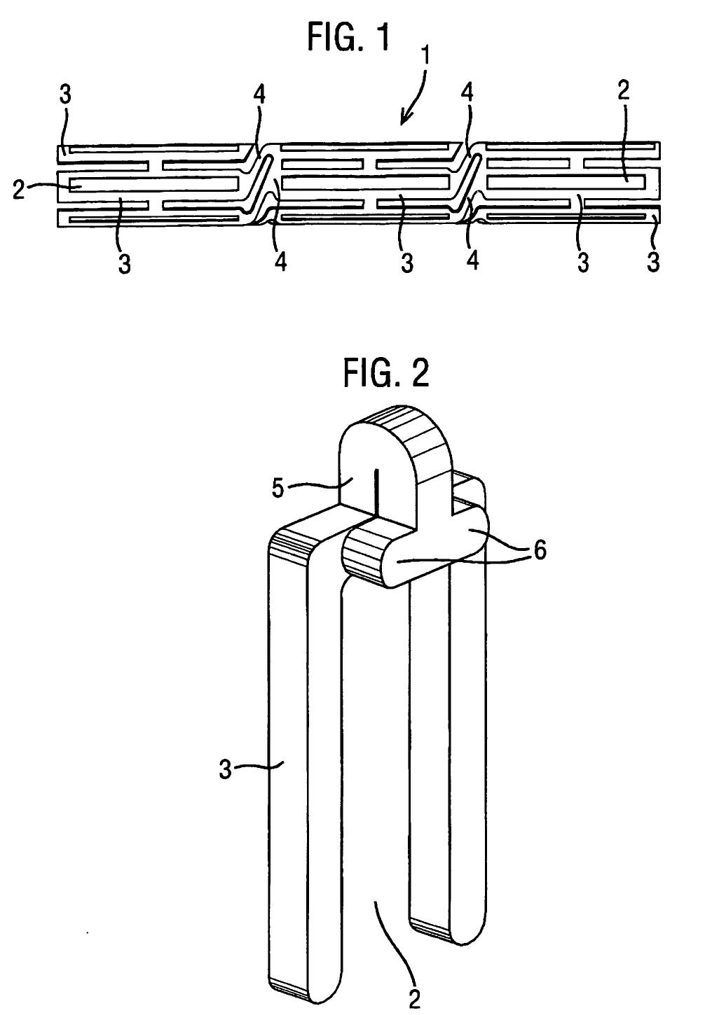

FIG. 1 shows a stent 1 that is of the balloon-expandable type. The stent 1 is formed from a hollow tube of, in this case, metal which is surgical steel or similar material that is capable of being inserted into a body lumen without adverse reaction. The stent 1 is formed by creating apertures 2 in the hollow tube that are formed by chemical or laser etching or similar material removal processes. This leaves a hollow tube which has a series of apertures 2 defined by struts 3. The struts may be formed so that they have bends 4 which allow for the stent 1 to be flexed as it is passed through a body lumen during the delivery process. In use, the stent 1 is delivered, in most cases, over an expandable balloon, so that when the stent is placed at its delivery site the balloon is expanded so that it fits within the appropriate body lumen and holds it open. The expandable balloon can then be deflated and removed. It will be appreciated that when the struts 3 are particularly narrow and when the overall diameter of the stent 1 is particularly small it can be difficult to view the stent by X-ray imaging as is required in order to ensure that a surgeon delivers the stent to the appropriate location in a body lumen.

The present invention overcomes this problem by providing one or more markers 5 on the stent 1. Example markers 5 are shown in FIGS. 2 to 6. In the example marker 5 of FIG. 2, the marker 5 is formed by bending over a strut 3 at one end of the stent 1 to produce a region of increased thickness that is radiopaque. The example marker of FIG. 2 has an increased area when viewed from the side by providing a “T” shaped configuration with arms 6.

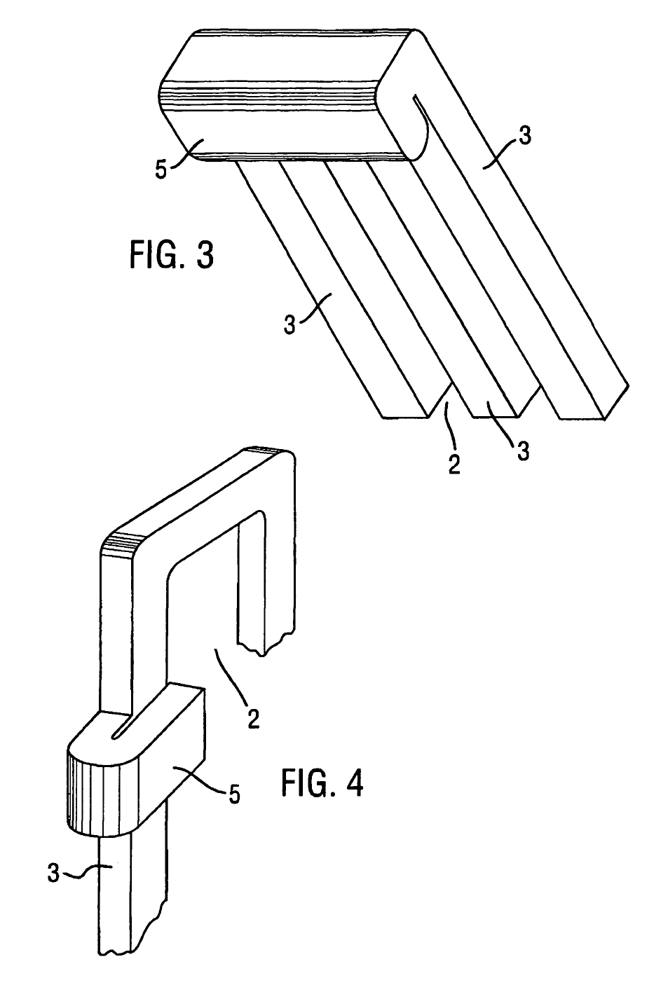

FIG. 3 shows a second example in which a marker 5 is formed by folding the connection point top of three struts 3 to form a region of increased thickness. Again, this example is formed at one end of the stent 1.

FIG. 4 shows an example in which a marker 5 is formed, again, by folding of a portion of a strut 3, but in this example in an intermediate area of the stent 1 away from its ends.

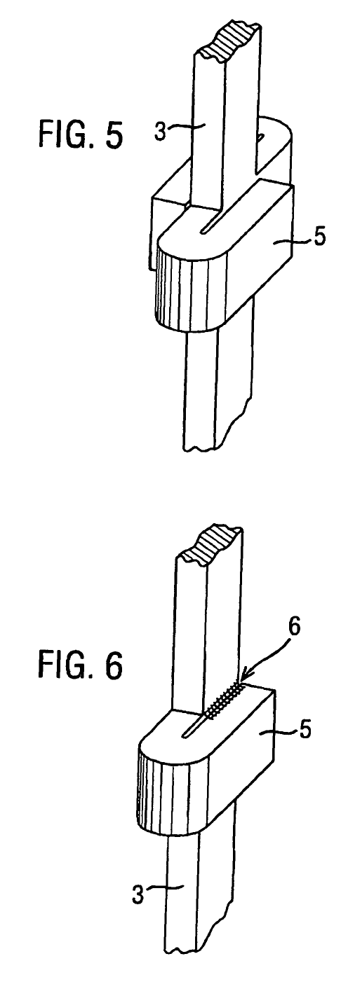

FIG. 5 shows a further example in which a marker 5 of treble thickness is produced by the folding over of a strut in two directions. The example of FIG. 5 shows a marker 5 formed in an intermediate position on the stent 1, although the same technique could be used at either end of the stent 1 with a similar effect.

It will be appreciated that a particular stent 1 may be formed using a combination of the above examples, and that in either the end or intermediate position examples more than one marker 5 may be positioned in any one region of the stent 1.

In all of the examples, it is preferable to secure the marker 5 by welding the folded portion to produce a joining weld 6 that holds the markers onto the strut 3 from which it is formed, so that fraying and snagging of the marker 5 does not occur.

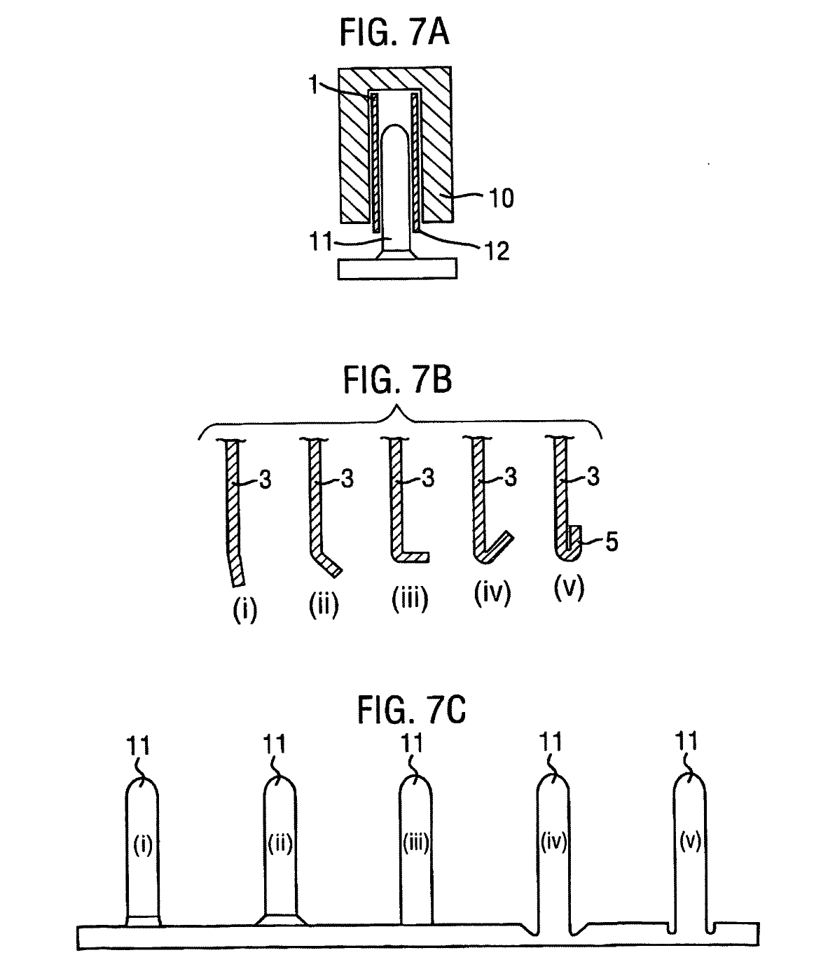

FIGS. 7A to 7C show an example of how example markers 5 of the earlier figures can be produced. A stent 1, in which apertures 2 have already been formed, is placed in a housing 10 which is in turn placed over a mandrel 11. By appropriate shaping of the tip of the housing 10 and the mandrel 11, relative movement of the housing 10 and mandrel 11 folds the exposed end 12 of the stent 1 to produce a marker 5. In many cases, to produce an accurate and reliable fold of the marker 5, a series of mandrels 11 (FIG. 7C) may be provided, with the stent 1 and possibly differing housing 10 for each step in combination being placed over each in turn to follow through steps (i) to (v) as shown in the combination of FIGS. 7B and 7C, the result at the end of step (v) being the production of the appropriate marker 5. This can, where appropriate, be followed by a welding step (not shown).

The description given above provides example implementation of this invention. Variations and modifications may become apparent to those skilled in the art that do not necessarily depart from the basis of this invention. The scope of legal protection given to this invention can only be determined by studying the following claims.

What is claimed is:

1. A stent comprising:
a tubular wall with apertures formed therein to allow for expansion of the stent; and
said stent including marker sections formed by folding at least one portion of the tubular wall to provide a region of increased thickness.

2. The stent as set forth in claim 1 wherein said marker sections are formed at the ends of the stent.

3. The stent as set forth in claim 1 wherein said marker sections are formed at an intermediate area of the stent.

4. The stent as set forth in claim 1 wherein said marker sections are formed by folding the at least one portion of the wall to the extent that an original outer surface of the portion is adjacent to an outer surface of the wall such that a two layer region of wall is provided as a marker section.

5. The stent as set forth in claim 1 wherein said marker sections are formed by folding the at least one portion of the wall into three layers.

6. The stent as set forth in claim 1 wherein said folded portions of the marker sections may be secured by welds.

7. The stent as set forth in claim 1 wherein said stent is expandable by outward pressure applied to the stent by an expandable balloon.

8. A method of manufacturing a stent including a tubular wall with apertures formed therein to allow for expansion of the stent, the method comprising the steps of:
providing a stent having a stent wall;
providing a die which is capable of presenting a series of inclined surfaces to at least one portion of the stent wall;
forming a contact between the at least one portion of the stent wall and the die; and
progressively folding the portion of the stent wall to provide a marker section of increased wall thickness by generating pressure on the contact.

9. The method as set forth in claim 8 wherein said die includes multiple components with a different inclined surface being provided by each component, such that the contact and folding steps further comprise the steps of:
presenting the stent to a first component of the die;
generating pressure at the stent contact to force the stent to take the shape of the component; and
repeating these steps at each component, in turn, to progressively form the folds of the marker sections.

10. The method as set forth in claim 9 wherein said components of the die include a series of protrusions, with the base of each protrusion comprising a molded region, wherein the shapes of these molded regions are sequentially shaped to progressively fold the wall region when the stent is pressed onto each protrusion in turn, with said method further comprising the steps of providing a housing to support the stent, and wherein the step of presenting the stent to the die further comprises the steps of:
placing the stent, surrounded by its housing, over one of the protrusions of the die;
introducing one end of the stent to the base of the protrusion;
generating pressure at the contact view to the housing such that the end of the stent is forced to take the shape of the base of the protrusion; and
removing the stent from the protrusion and placing the stent over the next protrusion.

11. The method as set forth in claim 8 further comprising the step of welding the folded portions of the marker sections in place.

12. The method as set forth in claim 8 wherein said stent is expandable by outward pressure applied to the stent by an expandable balloon.
